(19) United States
(12) Patent Application Publication
(10) Pub. No.: US 2024/0173094 A1
(43) Pub. Date: May 30, 2024

(54) SMART CABINET FOR EMERGENCY INTERVENTION

(71) Applicant: Hero TechLabs Inc., Shelton, CT (US)

(72) Inventors: Richard Imbruce, Westport, CT (US); Gregory R. Imbruce, Stamford, CT (US)

(21) Appl. No.: 18/522,956

(22) Filed: Nov. 29, 2023

Related U.S. Application Data
(60) Provisional application No. 63/428,655, filed on Nov. 29, 2022.

(51) Int. Cl. A61B 50/13; A61B 50/10; G16H 80/00

(52) U.S. Cl. CPC A61B 50/13; G16H 80/00; A61B 2050/105; A61B 2505/01

(57) ABSTRACT

A communications-equipped cabinet comprising a plurality of chambers, each chamber holding one or more emergency response items, is described. Each chamber has a door restricting access to the emergency response items contained therein. At least one door has a door monitor apparatus attached that sends a signal to a controller indicating that the respective door is no longer closed. The controller sends a signal that activates a monitor attached to the cabinet when the respective door is no longer closed.

### BACKGROUND

This application claims priority under 35 U.S.C. § 119 to U.S. Provisional Patent Application No. 63/428,655, filed on Nov. 29, 2022. The disclosure of that application is incorporated herein by reference.

The present disclosure relates to a communications-enabled cabinet holding medical treatment and emergency response items, equipment, and supplies. In particular, the present disclosure describes a cabinet that has multiple chambers for holding various pieces of medical and emergency response equipment and is adapted to establish an integrated notification-and-remote communications system between a person proximate to the cabinet (which besides the person in distress may also include first responders and “Good Samaritans”) and an emergency response entity, such as a hospital or fire department, wherein removal of pre-positioned equipment from the cabinet establishes a two-way communications link between that person and a professional emergency responder to instruct the person on using the equipment, items, or supplies, or to otherwise respond to an emergency, as well as to determine if the incident requires additional emergency services. In addition, the present disclosure relates to a communications-enabled cabinet adapted to provide non-emergency customer assistance functions, for example, to assist customers to select, locate, and/or use products at a retail sales establishment.

### BRIEF SUMMARY

Sometimes medical emergencies strike that would have been mitigated and/or averted were the right equipment accessible. As an example, for an individual in respiratory distress the presence of an emergency oxygen device nearby may be the deciding factor for survival, as the supplying of oxygen immediately to the victim during those first critical minutes would bridge the time gap between that medical event and the arrival of emergency medical services (EMS).

Addressing medical emergencies may also require that first aid or medical devices or items be provided to a victim as quickly as possible to stabilize the individual while awaiting EMS in an attempt to prevent further harm. For example, it may be vital that a victim with a breathing emergency receive oxygen as soon as possible—the cabinet of the present disclosure makes it safe and easy to access and use oxygen readily available during that period awaiting EMS to arrive on scene. Proper instruction on administering a first aid device or performing a life-saving procedure may be critical in such a situation and can be provided remotely by an emergency medical technician (EMT) via the instant cabinet and integrated display. The cabinet's communication system itself and/or an appropriate representative, can according to some embodiments of the disclosure, notify the appropriate emergency services entity upon occurrence of the medical emergency to expeditiously provide EMS with information regarding the event to maximize the likelihood of an optimal outcome.

### BRIEF DESCRIPTION OF THE DRAWINGS

These and other features and advantages of the various embodiments disclosed herein will be better understood with respect to the following description and drawings, in which like numbers refer to like parts throughout, and in which:

FIGS. 1-19 are elevation and perspective views of a communications-enabled cabinet according to embodiments of the disclosure.

FIG. 20 illustrates an exemplary block diagram of embodiments of the disclosure.

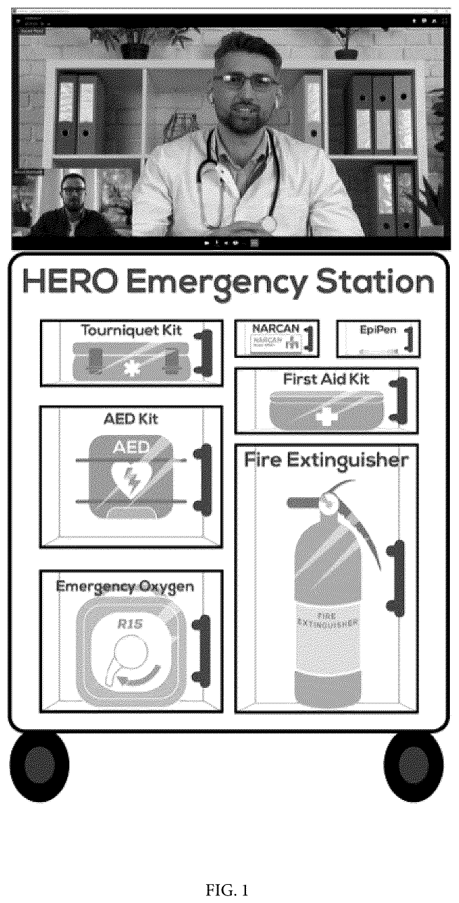

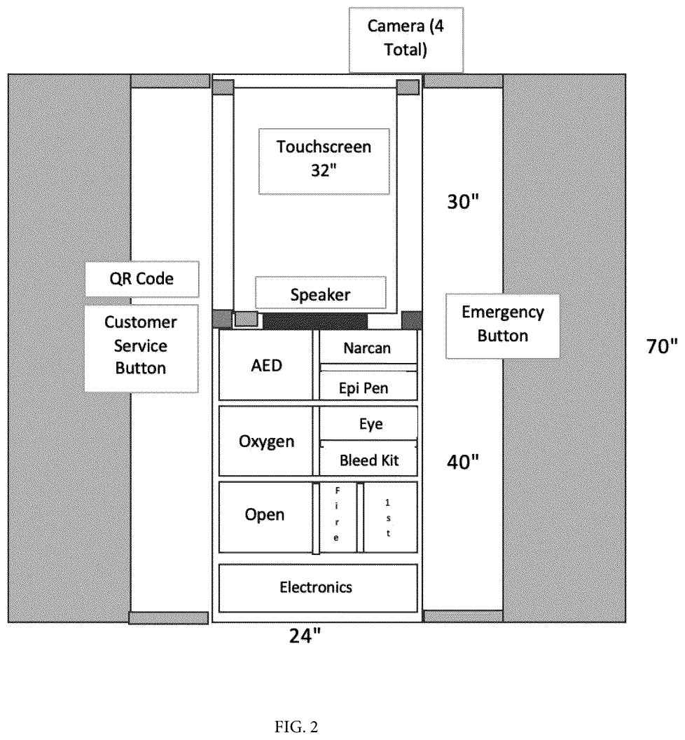

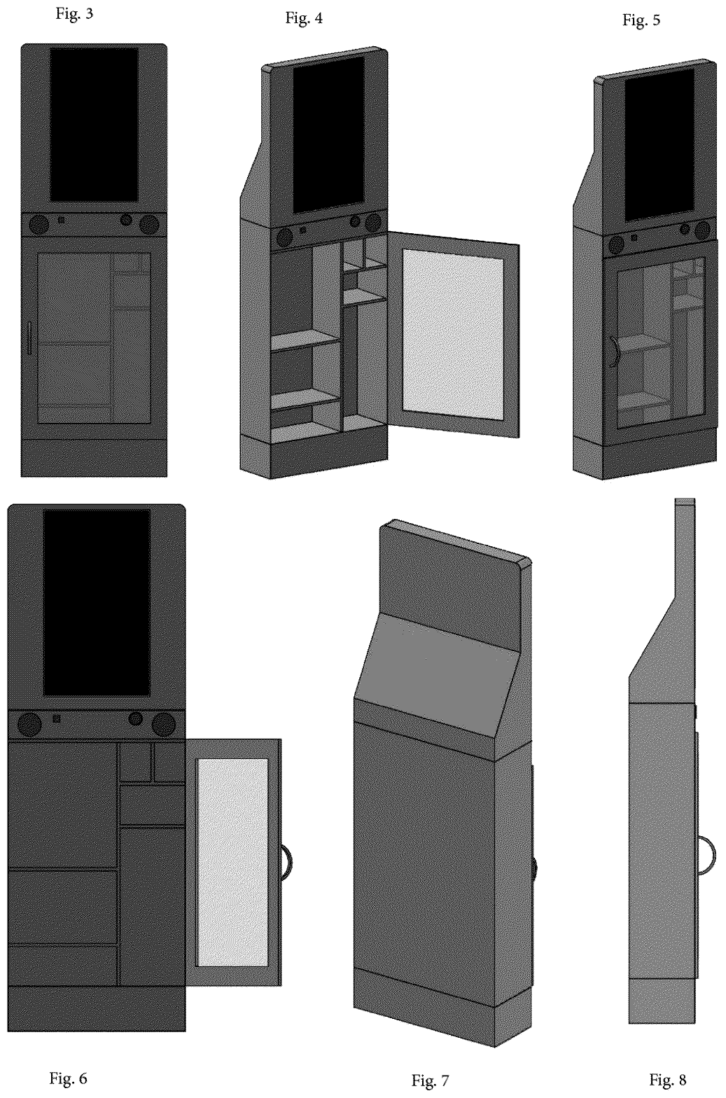

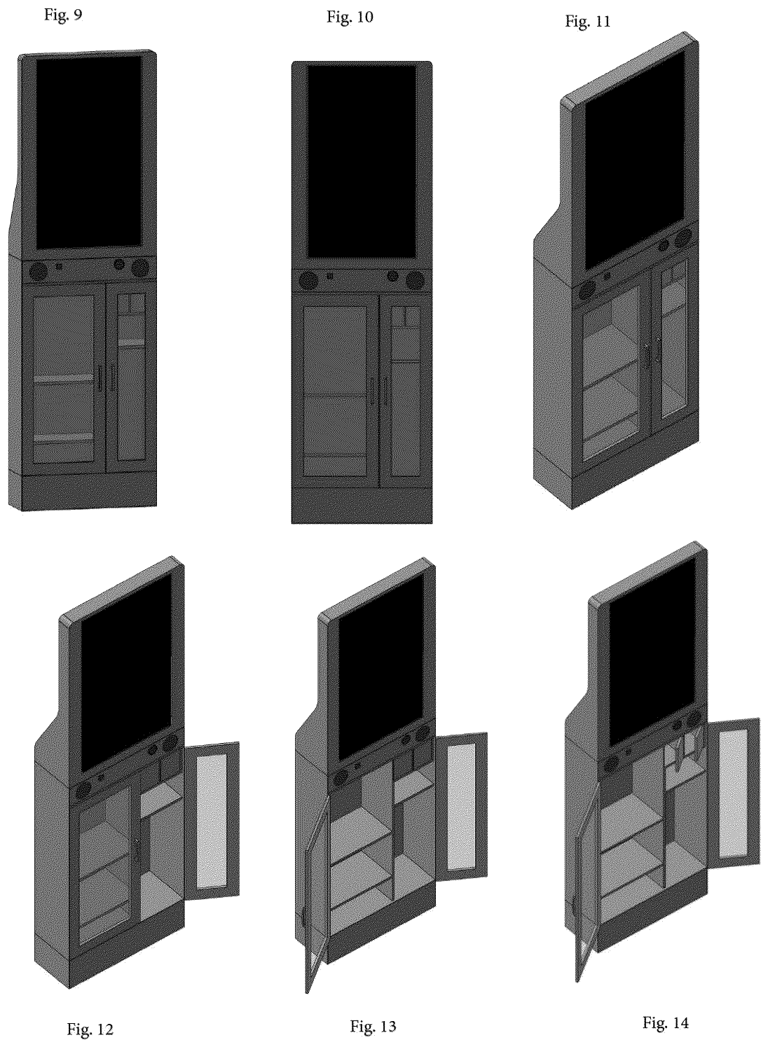

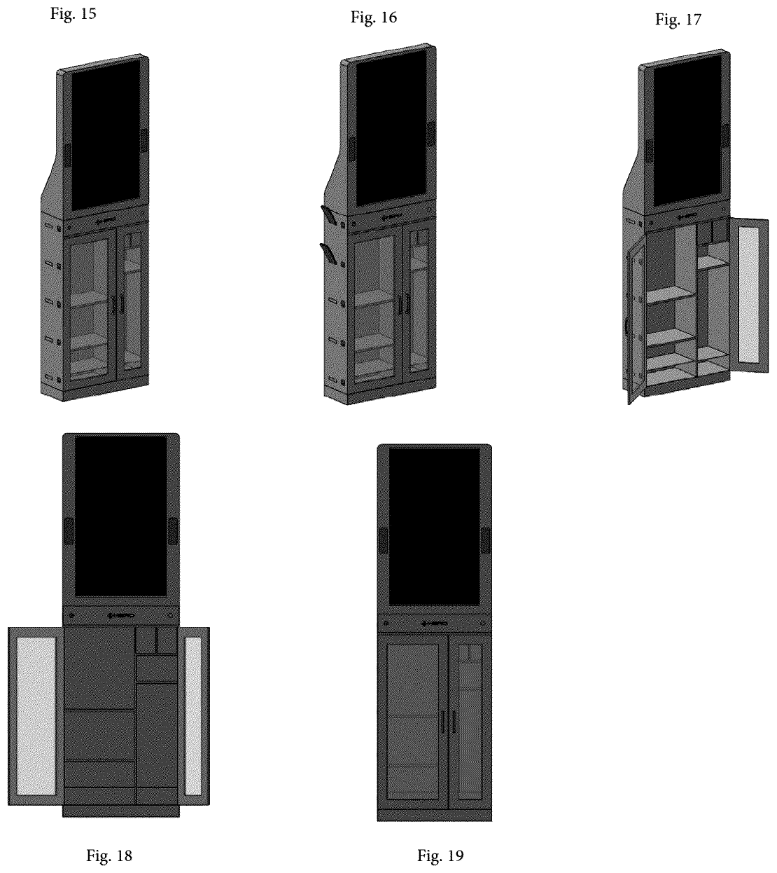

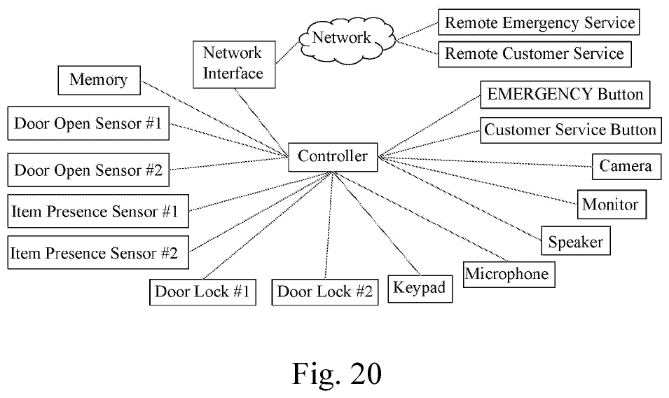

### DETAILED DESCRIPTION

The cabinet of the present disclosure, also known as “smart cabinet” or a “HERO Station,” is a communications-equipped cabinet for holding and accessing items to be used to respond to an emergency. The emergency response items may include medical, rescue, and first aid equipment and supplies. It is primarily meant to be a lifesaving solution available in any environment where there are relatively high densities of people in both public and private locations. Examples include airports or other mass transit facilities, schools, shopping malls, workplaces (e.g., office complexes, manufacturing plants), athletic stadiums, health clubs, venue halls, houses of worship—in short, anywhere people congregate that is not a hospital or other similarly equipped healthcare facility. According to some embodiments, the cabinet will have a Global Positioning System (GPS) or similar device and/or technology to provide its location.

As mentioned, the disclosure is directed to a specialized cabinet. The preferred embodiment of the cabinet is made of steel and/or aluminum and plastic, or similar materials, and, in one embodiment, has wheels for maneuverability and easy deployability. The cabinet contains several storage spaces or chambers for holding various medical, rescue, and first aid equipment and supplies (the preferred embodiment has eight storage spaces, but that number is not fixed).

The cabinet of the present disclosure may have one, two, or multiple doors covering the various storage spaces that grant or deny access to what is behind the respective door. In a preferred embodiment, each door is made of a transparent or semi-transparent material that allows an outside observer to see what is in the storage space. Alternatively, a door may be made of non-transparent material but have lettering on the outside indicating what equipment or item is in the respective storage space. Additionally, a door may be a combination of non-transparent material with a window that is a portion of the door; such doors may either have writing on the non-transparent portion identifying the contents or not, as the insides of the space are observable from outside.

If each storage space does not have its own door, and there are, for example, two primary doors, then there may exist select interior doors to enable such device or item to be either locked or unlocked, depending on which item is in a particular storage space. As can be appreciated, and as described further below, certain items stored in the cabinet of the present disclosure may require authorization and/or prescription from a medical professional before access and use is permitted. These doors that are capable of being locked are remotely controllable, i.e., an off-premises individual will be able to lock and unlock those doors electronically from a distance.

Each door has an arm to keep it propped open for easy access to and removal of desired equipment and items from the respective storage space. Moreover, each door has a door-open sensor to alert the facility—by email, short message service (sms), phone, or other form of electronic or digital notification—when a specific door has been opened and an item-presence sensor to detect if an item has been removed. According to one embodiment, no individual storage space will have more than one piece of medical/rescue/first aid equipment. According to another embodiment, one or more of the spaces is adapted to hold multiple items, e.g., multiple single-dose injection pens.

Below is an illustrative listing of the items and equipment included in the cabinet of the disclosure, but the list is not exhaustive. Those skilled in the art will recognize that other medical/rescue/first aid equipment would be suitable for inclusion. Next to each item or piece of equipment is an identification of whether the door for that storage space is locked or unlocked before the instance in which such equipment is needed and access is granted, as remotely determined by an appropriate representative.
Automated external defibrillator (AED)—unlocked door
Oxygen container (e.g., RapidOxygen™ R15)—unlocked door
Auto-injectable device (e.g., EpiPen®)—locked door
Fire extinguisher—unlocked door
First aid kit—unlocked door
Tourniquet/bleed kit—unlocked door
Dechoker—unlocked door
Fire blanket—unlocked door
NARCAN® (Naloxone HCl)—locked door

One or more of the above-listed items may have a tracking tag (e.g., radio-frequency identification, or RFID) attached. The cabinet may be equipped with an RFID tag reader, Bluetooth antenna, or other mechanism for determining not only whether a removed item has been returned, but also its pinpoint location on a digital map. According to one embodiment, once an item is removed from its associated storage space, an alert mechanism associated with the cabinet generates an alert signal to a service entity to place an automatic order to refill that specific item if it is not replaced in the storage space within 15 minutes. According to another embodiment, removal of certain equipment or items triggers a service-alert or replacement signal once the item is removed regardless of whether it is returned, e.g., where the equipment requires service after each operation (e.g., fire extinguisher). The cabinet of the present disclosure uses an RFID, Bluetooth, or similar communication device or system to detect when a device or item is removed from its specified storage space. The removal notification is generated immediately upon the device's or item's removal from the cabinet, while a usage notification is automatically generated if the same item is not returned to its storage space within one minute. In such instances, the cabinet's system may trigger an order for a replacement device or item given that the individual who took it out may have damaged or potentially rendered it unusable. According to one embodiment, the customer, administrator, manager, or employee of an entity with ownership or control of the cabinet has the ability to determine if the device or item was actually used and whether a replacement is required. A remote video camera associated with the cabinet may also enable the administrator to determine if any items appear to have been used or otherwise compromised for future usage.

A computer screen monitor or similar digital display is integrated into the top of the cabinet. While the size of the monitor is not critical, it should be of a dimension to allow displayed images of those in communication with the cabinet via the monitor to be easily viewed. The monitor may also be a touchscreen. In a preferred embodiment, the touchscreen monitor is attached to the cabinet with a tilt bracket to allow a user to select an ideal viewing position for the monitor. The monitor also interacts in combination with a camera, voice, and speaker communication system, as well as a controller, such as a microcontroller or CPU and other incorporated electronics. The monitor is activated when any door is opened, initiating live communication with an emergency-response entity. Another way in which to initiate communications concerning emergencies is to push the EMERGENCY button on the cabinet or an EMERGENCY button on the touchscreen. Once triggered by any of these methods, this cabinet will alert designated individuals by providing information including (i) the existence of an emergency, (ii) the location of the emergency, and (iii) the type of emergency, which is indicated by the specific item(s) removed (e.g., oxygen, AED, or fire extinguisher), in addition to determining which, if any, of the locked doors were remotely unlocked to give access to the protected materials therein.

The designated individuals alerted by the triggering event include an emergency responder or healthcare professional with skills appropriate to such emergency(ies) for the particular equipment door opened or item removed. This responder will appear on the integrated display to communicate with the user and/or victim to assist on the use of a particular stored device and/or assistance in attending to the emergency. The emergency representative is in place to not only immediately assist in the response to an emergency or terrorist attack, but also to act as a filter to determine if an event requires emergency services and, if so, whether such response involves internal and/or external emergency services (e.g., 911). The emergency representative, when appropriate, will alert 911 or the proper emergency services of the emergency type and pinpoint location, when available, so that such emergency service(s) can respond as quickly and efficiently as possible.

As previously described, the communications device and interface includes the video monitor, camera(s), microphone, and speaker(s) to facilitate a two-way communications link such as a videoconference between a user, such as a bystander at the scene of an emergency, and a remote emergency services professional. The professional can assist the bystander in addressing the emergency. According to one embodiment, the communications link enables the professional to control one or more of remotely operable locks to grant access to the bystander to access one or more pieces of equipment (at the professional's discretion) and/or any prescribed medications, such as an EpiPen® or NARCAN® dosage.

The cabinet and/or the individual pieces of equipment may include a GPS-enabled sensor to inform the remote professional of the distress location. In addition, beacon tags may be provided on each device to determine an exact location of the emergency in the event the device or item travels away from the cabinet itself. The system monitors the location where the equipment is taken (that is, to the site where a patient is suffering) and relays that information, either directly or via the communication system in the cabinet, to the emergency responder, as well as the system administrator.

Additionally, the cabinet may contain a chaos sensor to detect whether a number of people are rushing toward and/or moving quickly in the vicinity of the cabinet, which will inform the emergency responder of the potential existence of an emergency even before there is a physical triggering event by an individual.

The rear side of the cabinet contains an equipment compartment housing components necessary to or helpful for the cabinet's proper functioning. Included are (a) a central processing unit (CPU), (b) tracking tag (RFID) reader, (c) an RFID antenna, (d) a fan (for cooling equipment), (d) GPS locator (for identifying the cabinet's location and each of the included devices and items), (e) an electric plug strip, and (f) a backup battery to run the monitor/display, speakers, microphone, camera, fan, and beacon/antenna/reader. According to one embodiment, the cabinet is equipped with a temperature gauge to monitor and ensure that at least the computer equipment and oxygen unit are kept at room temperature, which is required for both. According to another embodiment, a heating and/or cooling mechanism is provided for one or more of the storage spaces to keep selected pieces of equipment or supplies within a selected storage temperature range.

While the principal purpose of the cabinet is emergency-related, a further feature of the device of the present disclosure is a remote customer service function. A user, upon registration with the user's cell phone number and/or email and subsequent login, could activate the system, for example, by touching a physical CUSTOMER SERVICE button or on the monitor's touch screen to cause the system to contact a customer service agent. In a retail setting, a customer could use the system to contact a customer service agent at a call center or even within the specific location for help in locating or selecting a product from the retail store. As one example, a customer in a large national-chain hardware store looking for a particular type of electric drill could go to a cabinet and push the CUSTOMER SERVICE button and be connected both visually and aurally with a sales agent who could answer the query. Similarly, in an airport, the system could put the user in contact with an airline booking agent for travel assistance. Each cabinet owned by a facility owner will also be networked, which will enable the facility owner to broadcast messages and content across all the cabinets owned. As an example of this networking capability, the same retail store in the prior example can broadcast emergencies in a specific store, or sales of a specific item across all stores. This network feature will also provide a platform for live in-store instructional sessions on how, for example, to install a door handle or replace kitchen floor tile.

The customer service and emergency function may be combined in one system or separated for individual functionality limited to emergency only or customer only.

A further feature of the instant disclosure is the capability to interface with other health data sensors, such as smart watches, to collect and analyze health data from users. In a preferred embodiment, the device of the present disclosure would be capable of integrating with a health watch consumer product by providing/forwarding the below health data for a number of measurements. To secure these measurements, the cabinet would have at least the following devices integrated: (a) a finger input for pulse oximeter and blood oxygen saturation, (ii) two metals plates for an electrocardiogram (ECG), (c) a heart rate monitor, and (d) a blood pressure measuring device (e.g., sphygmomanometer). Once a user records these health data, they can be collected, stored, shared, and/or sent electronically and/or digitally to a doctor, other health professional, or personal device (e.g., health consumer watch), or uploaded to a different site of the user's choosing, for present or future reference. Alternatively, the cabinet or remote HERO site could also analyze the results and provide a rating (e.g., a green, yellow, or red light) to apprise the user of a general view of the results, as well as various recommendations (e.g., an admonition to contact the user's doctor).

There are three applications (i.e., “apps”) for using the cabinet of the present disclosure that each support a different population/user, namely, (a) the user/patient of the station, (b) the customer (i.e., the facility where a cabinet is located), and (c) the entity that manufactures and administers the station (presently, HERO TechLabs Inc.).

For the user/patient application, the app will request a patient/user cell phone number for future contact or follow up, as well as to identify the emergency's location if that event is not proximate to the cabinet of the disclosure. The remote EMS representative will be, for example, a HERO employee or another provided by a third-party service, responsible for determining the scope of the emergency upon the initial communication and whether to escalate the event by contacting 911 with the pertinent information regarding the emergency, including, but not limited to, the location and type of emergency. This app will also allow users to register on the app or at the cabinet to store, access, use, and track health-related data and provide such information to the user's doctor or other medical professional or organization, upon the user's request. The personal data will include basic contact information, including name, address, cell phone, email, date of birth, gender/gender identification, general health status, primary healthcare provider (with contact information), in addition to other information to begin a profile and establish an account.

This app will also contain informational and educational materials concerning the cabinet and its capabilities, as well as explaining each device or item included in the cabinet. Other features, such as navigation through the facility where a particular cabinet is located and advising of promotional items, will likewise be available. The display integrated into the cabinet will also be used for advertising purposes, based upon the respective cabinet owner's preferences.

The facility management application will provide a notification management system for email, sms, phone, or other form of electronic or digital notification concerning the status of the cabinets, their included devices and items, and their use. Reports that will be available through this app include status of each cabinet and whether the requisite items are present in the cabinet. Additionally, the app will report if an individual item in a cabinet is nearing, at, or past its respective expiration date, where applicable. The app will also report on whether there is a battery power issue with a particular AED in a cabinet, or adequate pressure in the fire extinguisher upon use. The app will report when a consumable has been removed and when it has been replaced and will contain an alert if there is a gap of more than fifteen minutes between removal and replacement. Finally, the app will alert if there is a need for a service call or replacement of a consumable device or item.

The third app is the HERO Global Remote Center Application. This app will report usage data worldwide for all cabinets and will provide access to all aspects of each unit (including the stored devices and items), including, for example, the respective status of the cabinet's components, such as cabinet doors and the backup battery, and, where available, the devices and items stored in the cabinet. Further, the app will show the location of each unit and identify at which customer's facility the unit is located. The app will further show the status of each cabinet and whether there is an event occurring, as well as whether EMS or other emergency services have been alerted of such event, and a log and recording of such interaction. Billing management will also be available on the app.

The above description is given by way of example, and not limitation. Given the above disclosure, one skilled in the art could devise variations that are within the scope and spirit of the disclosure disclosed herein. Further, the various features of the embodiments disclosed herein can be used alone, or in varying combinations with each other, and are not intended to be limited to the specific combination described herein. Thus, the scope of the claims is not to be limited by the illustrated embodiments.

We claim:

1. A communications-equipped cabinet, comprising:
a plurality of chambers formed in the cabinet for holding one or more emergency response items;
one or more doors, wherein each of the one or more doors is associated with a respective one or more of the plurality of chambers and adapted to be moved away from a closed position;
a controller;
a door monitor apparatus in signal communication with the controller and coupled with at least one of the one or more doors, the door monitor sending a signal to the controller when the one door is moved away from the closed position communicating an identity of the one door and that the one door has been moved from the closed position; and
a monitor attached to the cabinet and in signal communication with the controller, wherein the controller activates the monitor in response to the one door moving away from the closed position.

2. The cabinet of claim 1, further comprising a camera in signal communication with the controller and adapted to capture an image of an area proximate to the cabinet;
a speaker in signal communication with the controller;
a microphone in signal communication with the controller; and
a two-way communications system in signal communication with the controller, wherein the controller activates the speaker and the microphone in response to the one door moving away from the closed position.

3. The cabinet of claim 2, wherein the two-way communication system is adapted to establish a communications link with an emergency-response entity.

4. The cabinet of claim 1, further comprising a presence-detection apparatus connected with at least one of the chambers and in signal communication with the controller, wherein the at least one chamber removably holds a selected one of the emergency response items, and wherein the presence-detection apparatus sends an item removed signal to the controller in response to removal of the selected emergency response item from the at least one chamber.

5. The cabinet of claim 4, wherein the controller determines an emergency type based on the item removed signal.

6. The cabinet of claim 3, further comprising an activation button operable by a person and in signal communication with the controller, wherein, in response to operation of the activation button, the communications link is established and the monitor, camera, and speaker are activated to enable communication between the person and the emergency-response entity.

7. The cabinet of claim 1, wherein the one door is adapted to be moved away from an open position to the closed position, wherein, when the one door is moved to the closed position, the door monitor apparatus communicates to the controller the identity of the one door and that the one door has been moved to the closed position.
